(19) United States
(12) Patent Application Publication
(10) Pub. No.: US 2024/0407822 A1
(43) Pub. Date: Dec. 12, 2024

(54) ENDOSCOPIC OR ENDOBRONCHIAL TUMOR TREATMENT SYSTEMS AND METHODS

(71) Applicant: Mayo Foundation for Medical Education and Research, Rochester, MN (US)

(72) Inventors: Barham K. Abu Dayyeh, Rochester, MN (US); Jason A. Tri, Rochester, MN (US); Samuel J. Asirvatham, Rochester, MN (US); Suraj Kapa, Rochester, MN (US); Ryan M. Kern, Rochester, MN (US); John J. Mullon, Rochester, MN (US)

(21) Appl. No.: 18/697,223

(22) PCT Filed: Sep. 27, 2022
(86) PCT No.: PCT/US2022/044828

§ 371 Date: Mar. 29, 2024

Related U.S. Application Data
(60) Provisional application No. 63/250,543, filed on Sep. 30, 2021.

(51) Int. Cl. A61B 18/00; A61B 10/04; A61B 34/10

(52) U.S. Cl. CPC A61B 18/00; A61B 10/04; A61B 2010/045; A61B 2018/00083; A61B 2018/00184; A61B 2018/00541; A61B 2018/00613; A61B 34/10; A61B 2034/104

(57) ABSTRACT

Systems and methods can be used endoscopically to treat tumors in mediastinal, abdominal, and peri-rectal spaces. For example, this document describes systems and methods for endoscopic delivery of non-thermal pulsed electrical field or electroporation to such tumors. In addition, the systems and methods prime the immune system by re-introducing obtained and processed tissue or aspirates back to the local tumor environment after ex-vivo denaturing and emulsification of the tumor tissue or other immune enhancing substances such a monoclonal or multifunctional antibodies. The re-introduction of the processed tumor cells or immune enhancing substances will create an antigenic primed environment to the enhanced immune response generated by the pulsed electrical field. This will harness the power of the immune system as an ablative modality to the local tumor while systemically targeting metastatic disease.

### CROSS-REFERENCE TO RELATED APPLICATIONS

This application claims the benefit of U.S. Provisional Application Ser. No. 63/250,543, filed Sep. 30, 2021. The disclosure of the prior application is considered part of (and is incorporated by reference in) the disclosure of this application.

### BACKGROUND

### 1. Technical Field

This document relates to systems and methods for endoscopically or endobronchially treating tumors. For example, this document relates to robotic, electronic, and/or manual systems and methods for endoscopic or endobronchial delivery of pulsed electrical field ablation and/or electromechanical field ablation. In some embodiments, one or more processed immunogenic (immune stimulating or immune-suppressing or a combination) substances can optionally be delivered to treat tumors in the mediastinal cavity, abdominal cavity, lung, liver, pancreas, adrenal gland, celiac ganglia, prostate, omental or visceral fat, and peri-rectal/peri-colonic spaces.

### 2. Background Information

Currently, tumors in mediastinal and abdominal locations are conventionally treated using heat-mediated ablation or chemotherapy. In some cases, heat-mediated ablation can be inefficient and can cause collateral damage to surrounding organs and tissue. Some tumors, such as pancreas adenocarcinoma, have a robust fibrotic capsule shielding them from the effects of systemic chemotherapy.

Surgical resection with adjuvant chemotherapy is generally considered the best treatment option for long-term survival. However, less than 20% of patients present with disease amenable for resection because of vascular involvement, and even in this subset of patients, overall survival after successful resection and adjuvant therapy is extremely poor.

### SUMMARY

This document describes systems and methods for endoscopically or endobronchially treating tumors. For example, this document describes systems and methods for endoscopic delivery of non-thermal pulsed electrical field ablation, radiofrequency ablation, cryoablation, high intensity focused ultrasound ablation, photo dielectric ablation alone or in combination with concurrent or subsequent injection of an immunogenic substance such as processed core tissue from the tumor with an immune stimulant (such as, but not limited to, toll-like receptor agonists, saponin based adjuvants, stimulant of interferon gene, double stranded RNA analogues, imiquimod, or others) and substances to alter the immune suppressive microenvironment (such as, but not limited to, scavenger or antagonists of immunes suppressive cytokines, check point inhibitors, monoclonal antibodies [such as antiCD25], multifunctional antibodies, etc.) to locally prime the tumor for enhance immunogenic anti-tumor effect that destroys the tumor locally or at distance metastatic site. Such tumors can include, but are not limited to, malignant metastasis to lymph nodes, liver malignant lesions (primary to metastasis), pancreas mass lesions (solid and cystic), adrenal lesions, mediastinal and retroperitoneal lesions, lung mass lesions, and intra/extrahepatic biliary lesions.

In one implementation, an endoscopic or endobronchial tumor treatment system includes: an ultrasound probe comprising a shaft and an ultrasonic transducer attached to a distal end of the shaft, the shaft defining a working channel; a sheath defining a first lumen and a second lumen, the sheath being slidably disposable within the working channel and extendable from a distal end of the working channel; a needle slidably disposed within the first lumen, the needle defining a lumen and configured to be a cathode; and an electrode lead slidably disposed within the second lumen and configured to be an anode. In some embodiments, the working channel and/or the sheath includes a structure that causes the sheath and/or needles to emerge distally at a non-zero angle relative to the longitudinal axis of the shaft. The structure can be selectively controllable in some embodiments by an elevator function that is controlled mechanically or electronically or robotically, or in other embodiments the structure can be an immovable mechanical structure.

Such an endoscopic or endobronchial tumor treatment system may optionally include one or more of the following features. The electrode lead may be a stylet-driven flexible lead that is configurable by an internal stylet. Accordingly, the stylet can be used to configure and/or steer the flexible lead along one or more curves, angles, directions, etc. In some embodiments, the stylet can be manually controlled to configure selectively the flexible lead as the clinician desires. The electrode lead may include one or more contact electrodes, two or more contact electrodes, and/or three or more contact electrodes, without limitation. In some embodiments, the electrode lead can take a helical configuration to allow it to penetrate and stabilize in the desired tissue target. The needle may be configured to obtain a tumor tissue sample and to inject processed tissue or other immune enhancing substances into a tumor.

In another aspect, this disclosure is directed to another endoscopic or endobronchial tumor treatment system that includes: an ultrasound probe comprising a shaft and an ultrasonic transducer attached to a distal end of the shaft, the shaft defining a working channel; a needle slidably disposed within the working channel, the needle defining a lumen and configured to be a cathode; a collar adapter attached to the shaft of the endoscope and defining a lumen; and an electrode lead slidably disposed within the lumen of the collar adapter and configured to be an anode. In some embodiments, an anchor mechanism, such as a deployable screw, is included to secure the system at an anatomical location to be treated. For example, in some embodiments a deployable screw can be extended to penetrate tissue at the location to be treated so as to anchor and spatially maintain the system while the needle(s) are used to deliver electroporation and/or substances.

Such an endoscopic or endobronchial tumor treatment system may optionally include one or more of the following features. The system may include an outer insulative covering on the needle except for on a distal end portion of the needle. The electrode lead may be a stylet-driven flexible lead that is configurable by an internal stylet. The electrode lead may include one or more contact electrodes, two or more contact electrodes, or three or more contact electrodes, without limitation. The needle may be configured to obtain a tumor tissue sample and to inject processed tissue or other immune enhancing substances into a tumor.

In another aspect, this disclosure is directed to another endoscopic or endobronchial tumor treatment system that includes: an ultrasound probe comprising a shaft and an ultrasonic transducer attached to a distal end of the shaft, the shaft defining a working channel; a needle slidably disposed within the working channel, the needle defining a lumen and configured to be a cathode; a collar adapter attached to the shaft of the endoscope and defining a first lumen, a second lumen, and a third lumen; a first electrode lead slidably disposed within the first lumen of the collar adapter and configured to be an anode; a second electrode lead slidably disposed within the second lumen of the collar adapter and configured to be an anode; and a third electrode lead slidably disposed within the third lumen of the collar adapter and configured to be an anode.

Such an endoscopic or endobronchial tumor treatment system may optionally include one or more of the following features. The system may also include an outer insulative covering on the needle except for on a distal end portion of the needle. The the first, second, and third electrode leads may each be stylet-driven flexible leads that are configurable by an internal stylet. The first, second, and third electrode leads may each comprise one or more contact electrodes, two or more contact electrodes, or three or more contact electrodes, without limitation. The needle may be configured to obtain a tumor tissue sample and to inject processed tissue or other immune enhancing substances into a tumor.

In another aspect, this disclosure is directed to another endoscopic or endobronchial tumor treatment system that includes: an ultrasound probe including a shaft and an ultrasonic transducer attached to a distal end of the shaft, the shaft defining a working channel; an anode electrode slidably disposed within the working channel, the anode electrode defining a lumen and configured to be an anode; and an insulated needle slidably disposed within the lumen of the anode electrode, the needle defining a lumen and configured to be a cathode. In some embodiments, certain components of the system are embodied in physically separate devices, rather than being integrated in a single assembly.

In another aspect, this disclosure is directed to another endoscopic or endobronchial tumor treatment system that includes: an ultrasound probe including a shaft and an ultrasonic transducer attached to a distal end of the shaft, the shaft defining a working channel; an anode electrode attached to the distal end of the shaft, the anode electrode defining an opening and configured to be an anode; and an insulated needle slidably disposed within the working channel and extending through the opening of the anode electrode, the needle defining a lumen and configured to be a cathode.

In another aspect, this disclosure is directed to a method of treating a tumor. The method includes: providing any one of the endoscopic or endobronchial tumor treatment systems as described herein; delivering, using the anode and the cathode and while under ultrasonic visualization provided by the ultrasonic transducer, a pulsed electric field to the tumor; and injecting, using the needle, processed tumor cells or other immune enhancing substances into the tumor.

Particular embodiments of the subject matter described in this document can be implemented to realize one or more of the following advantages. First, using the systems and methods described herein, the treatment of tumors is enhanced in at least two ways. Pulsed electrical field induced uniform cell death without heat diffusion/dissipation or coagulative necrosis seen in heat-based methods. This can reduce tumor load in the area exposed to the electrical field, but also maximize the release of immunogenic tumor cell and substances that are not damaged or denatured by heat (such as damage associated molecular pattern) to start a robust immune reaction and active the adaptive immune system to develop anti-bodies and killer t-cell that attacks the tumor locally or at a distant site. Simultaneous, or in sequence, the system enables obtaining and/or re-introducing reprocessed tumor samples back to the local tumor environment to enhance the immunogenicity of the tumor with additional substances as described above to modulate the local tumor environment and generate an immune response that enhances tumor treatment locally and system wide, targeting metastatic disease.

Second, tumors in mediastinal and abdominal locations can be treated using the devices and methods provided herein in a minimally invasive fashion using the devices and methods provided herein. Such minimally invasive techniques can reduce recovery times, patient discomfort, and treatment costs.

Third, using the systems and methods described herein, tumor cells can be destroyed while avoiding the collateral damage to surrounding organs and tissue that can occur as a result of thermal ablation treatments. For example, hybrid transducers can be delivered via endoscopy, bronchoscopy, laparoscopy, or through the vascular system capable of administering a synergistic ultrasound mechanical forces and pulsed electrical field, that can enhance the ablative effects, therapeutic field depth, and decrease collateral damage of each modality alone.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described herein. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description herein. Other features, objects, and advantages of the invention will be apparent from the description and drawings, and from the claims.

### DESCRIPTION OF THE DRAWINGS

FIG. 1 is a perspective view of an example ultrasound guided endoscopic or endobronchial tumor treatment system in accordance with some embodiments provided herein.

FIG. 2 is a perspective view of the system of FIG. 1 in another configuration.

FIG. 3 is a perspective view of another example ultrasound guided endoscopic or endobronchial tumor treatment system in accordance with some embodiments provided herein.

FIG. 4 is a perspective view of the system of FIG. 3 in another configuration.

FIG. 5 is a perspective view of another example ultrasound guided endoscopic or endobronchial tumor treatment system in accordance with some embodiments provided herein.

FIG. 6 is a perspective view of the system of FIG. 5 in another configuration.

FIG. 7 is a perspective view of another example ultrasound guided endoscopic or endobronchial tumor treatment system in accordance with some embodiments provided herein.

FIG. 8 is a perspective view of another example ultrasound guided endoscopic or endobronchial tumor treatment system in accordance with some embodiments provided herein.

FIG. 9 is a perspective view of another example ultrasound guided endoscopic or endobronchial tumor treatment system in accordance with some embodiments provided herein.

FIG. 10 illustrates another example electrode lead that can be used for the ultrasound guided endoscopic or endobronchial tumor treatment systems in accordance with some embodiments.

FIG. 11 is a flowchart of an example method for pre-procedure planning that can be used in conjunction with the ultrasound guided endoscopic or endobronchial tumor treatment systems described herein.

FIG. 12 is an example of an electroporation modeling illustration.

FIG. 13 is an example of another electroporation modeling illustration.

Like reference numbers represent corresponding parts throughout.

### DETAILED DESCRIPTION

The field of endobronchial and endoscopic ultrasound offers reliable access to multiple mediastinal and abdominal structures that are difficult to access surgically. Endobronchial and endoscopic ultrasound provides the opportunity to treat a variety of malignant and pre-malignant etiologies.

This document describes systems and methods for ultrasound guided endoscopic or endobronchial tumor treatment system in mediastinal and abdominal spaces. For example, this document describes robotic, electronic, and/or manual systems and methods for endoscopic delivery of non-thermal electroporation or pulsed electrical field at different frequencies to such tumors. This document also describes that the systems can also be used for endoscopic delivery of radiofrequency ablation, cryoablation, ultrasound ablation, and/or photo dielectric ablation. In addition, the systems and methods prime the immune system by re-introducing obtained and processed tissue with or without other immune stimulating or enhancing substances back to the local tumor environment after ex-vivo denaturing and emulsification of the tumor tissue. The re-introduction of the processed tumor cells will create an antigenic primed environment to the enhanced immune response generated by the pulsed electrical field or other combination modalities introduced by the system; such as cryoablation, high frequency ultrasound, or sonoporation. This will harness the power of the immune system as a non-thermal ablative modality to the local tumor while systemically targeting metastatic disease.

In some examples, the types of tumors in mediastinal and abdominal spaces that are treatable using the systems and methods described herein can include, but are not limited to, malignant metastasis to lymph nodes, liver malignant lesions (primary to metastasis), pancreas mass lesions (solid and cystic), adrenal lesions, mediastinal and retroperitoneal lesions, lung mass lesions, peri-rectal, peri-colonic, and intra/extrahepatic biliary lesions.

Using a pulsed electric field (e.g., non-thermal electroporation) administered alone or in-combination with other modalities such as cryoablation or sonoporation through the system, reversible or irreversible nanoscale defects in the tumor cell membrane are induced to the area exposed to the electrical field, leading to cell death through apoptosis and or necrosis. Such electroporation is based on the pulsatile application of electric energy delivered between two or more electrodes that are introduced in and around the tumor (e.g., voltage: 1V to 20 kV with a pulse duration: e.g., 100 ns-300 us, or 20 ns-5 ms).

Due to its primarily non-thermal mechanism of action, electroporation leaves supporting extracellular matrix structures unaffected, preserving the structural tissue integrity of inlaying and nearby structures like vessels and bile ducts. This makes the technique ideal for the ablation of diffusely growing malignancies that surround these structures or close to critical structures such as major blood vessels. Two characteristics of electroporation make the technique a good treatment modality for the induction of a systemic antitumor immune response. First, electroporation-induced cell death and reduced tumor load may lead to a simultaneous release of immunogenic tumor cell remnants and a reduction in tumor-associated immunosuppression. Second, because the larger vessels remain intact, primed and recruited effector T cells can be enabled to infiltrate the lesion.

The systems described herein are compatible with currently available endobronchial or endoscopic ultrasound probes, and therefore pass through the working channel of these articulating instruments. The systems described herein includes a central needle that is capable of obtaining core or processed tissue from the tumor, as well as re-introducing the processed tissue with or without other immune enhancing substances back to the local tumor environment after denaturing and emulsification of the tumor tissue is complete. The central needle can also be used for the introduction of adjunct ablation or immune-stimulatory modalities such cryoablation. In addition, the systems described herein include at least one anode and one cathode that can be placed into the tumor to deliver the pulsed electrical field. The systems described herein are also compatible with currently available electroporation, or pulsed electrical field generators.

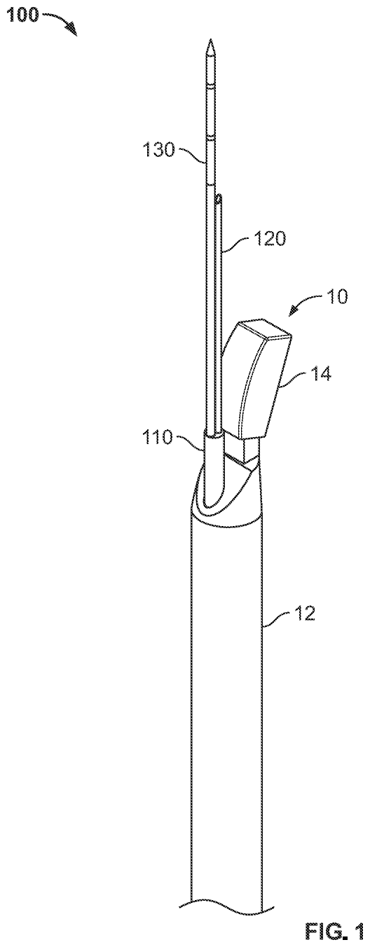

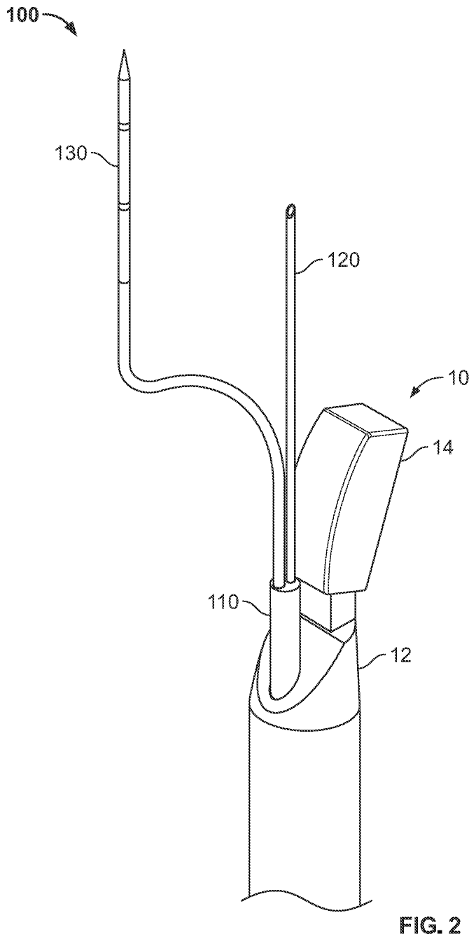

FIGS. 1 and 2 illustrate an example endoscopic tumor treatment system 100. Broadly speaking, the endoscopic tumor treatment system 100 includes an ultrasound probe 10, a sheath 110, a needle 120, and an electrode lead 130.

The endoscopic tumor treatment system 100 can be navigated within a patient to a target location. For example, in some cases the endoscopic tumor treatment system 100 can be navigated within areas of the body of a patient such as, but not limited to, an esophagus, stomach, colon, a trachea, or bronchus of the patient to deliver therapy to tumors in mediastinal or in the gastrointestinal traction or surrounding structures to deliver therapy. This can be performed while using visualization provided by the ultrasound probe 10. This probe 10 can be coupled with additional capabilities beyond visualization, such as delivery of different ultrasound waves of different mechanical indexes ranging from 0.01 to 10 and/or focused high-frequency ultrasound to alter the treated tissues characteristics for enhanced immune response.

The therapy delivered to tumors using the endoscopic tumor treatment system 100 can include at least two types of treatments. First, using the needle 120, processed tumor cells or immune enhancing substances can be injected into the tumor. Second, using the electrode lead 130 as an anode and the needle 120 as a cathode, pulsed electrical field electroporation can be delivered to the tumor. In some cases, the pulsed electrical field electroporation can be non-thermal electroporation. In some cases, the central needle 120 can also deliver adjunct ablation modalities such as cryoablation or other types of ablation as described herein. In addition, in some embodiments the system 100 can be used to deliver a low RF current to the area of interest to measure impedance of the tissue. This tissue impedance can be used for multiple reasons, such as to match the impedance that the electroporation generator delivers (e.g., the same impedance of the tissue and DC packet (PEF energy)), to determine electroporation success (e.g., if impedance drop meets a target it can indicate good ablation lesion), and/or to integrate into a impedance mapping system that can display the device and position within the body. In some embodiments, a CT scan or an external cross-section digital registration system can be integrated into the mapping system to guide ablation. This will allow the ability to lower fluoroscopy time and integrate electroporation modeling (as described further below).

The ultrasound probe 10 comprises a shaft 12 and an ultrasonic transducer 14 attached to a distal end of the shaft 12. The shaft 12 defines at least one working channel. The shaft 12 and/or ultrasonic transducer 14 can be deflectable to facilitate steering and/or directing of the ultrasound probe 10.

In the depicted embodiment, the sheath 110 is slidably disposed within the working channel of the shaft 12. The sheath 110 is distally extendable from a distal end of the working channel. The sheath 110 defines at least a first lumen and a second lumen.

The needle 120 is slidably disposed within the first lumen of the sheath 110. The needle 120 includes a tip configured for puncturing tissue. The needle 120 defines a lumen that is used for obtaining core tissues or fine needle aspirates from the tumor, and/or for injecting processed tumor cells or other immune enhancing substances into the tumor such as monoclonal or multifunctional antibodies to the local tumor environment with or without adjunct ablation modalities such as cryoablation. The needle 120 is configured to be a cathode that functions in cooperation with the electrode lead 130 to deliver pulsed electrical field or electroporation energy to a target tissue.

The electrode lead 130 is a flexible, and/or rotatable, and/or configurable lead that includes one or more contact electrodes that are spaced apart from each other. In some embodiments, three or more electrodes are included on the electrode lead 130. The contact electrode(s) on 130 can be activated individually, simultaneously, in sequence, or in tandem to generate the desired electrical field.

In some embodiments, the electrode lead 130 is a stylet-driven flexible lead that is configurable (e.g., steerable) in response to manipulation of an internal stylet. In some embodiments, the stylet within the electrode lead 130 is a rigid linear member. Then, when the stylet is withdrawn, the electrode lead 130 can reconfigure to its natural, unconstrained shape (e.g., the curved shape shown in FIG. 2, or any other desired natural shape). The electrode lead 130 can also be steered by controlling the position of the stylet in the electrode lead 130. That is, by partially withdrawing the stylet from the electrode lead 130, the electrode lead 130 can partially reconfigure to its natural shape. By controlling the extent to which the stylet is withdrawn, the electrode lead 130 can be shaped and steered.

FIG. 2, in particular, shows the endoscopic tumor treatment system 100 in a deployed state. In the deployed state, the electrode lead 130 and the needle 120 are both in position to contact tissue so that pulsed electrical field electroporation can be delivered to a target tissue (e.g., tumor, lesion, etc.) for therapeutic purposes. In addition, the needle 120 can be operated to obtain core tissue or fine needle aspirates from the tumor and/or for injecting processed tumor cells or other immune enhancing substances into the tumor. In addition, adjunct ablation modalities can be administered from this central needle 120, such as cryoablation. These operations can be performed under ultrasonic visualization using the ultrasound probe 10.

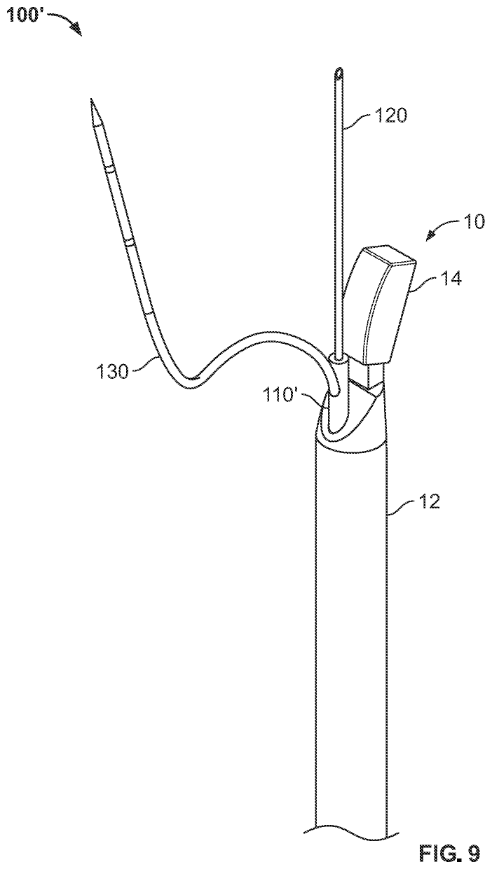

FIG. 9 shows an alternative endoscopic tumor treatment system 100′ that includes an alternative sheath 110′. In the depicted embodiment, the sheath 110′ has a lumen in which the electrode lead 130 is slidably disposed, and that terminates at a side port through which the electrode lead 130 exits. The sheath 110′ also includes an end port through which the needle 120 exits.

The lumen and its side port of the sheath 110′ can be configured to direct, deflect, or steer the electrode lead 130 to exit the sheath 110′ at a non-zero angle relative to the longitudinal axis of the sheath 110′ and the shaft 12 (e.g., as illustrated in FIG. 9). The exit angle defined between the sheath 110′ and electrode lead 130 can be in a range between 0° and 30°, or between 20° and 50°, or between 40° and 70°, or between 60° and 90°, without limitation. In some embodiments, the exit angle is adjustable by the clinician operator of the endoscopic tumor treatment system 100′. In some iterations, the angle or the exiting electrode is adjusted using an elevator or flap mechanism that is externally controlled mechanically, hydraulically, electrically, or robotically. In some embodiments, the lumen and its side port of the sheath 110′ can eliminate the need for a stylet within the electrode lead 130.

While the depicted embodiment of the sheath 110′ includes a single side port, in some embodiments the sheath 110′ can include two side ports, three side ports, four side ports, five side ports, six side ports, or more. Each side port can be associated with an individual lumen.

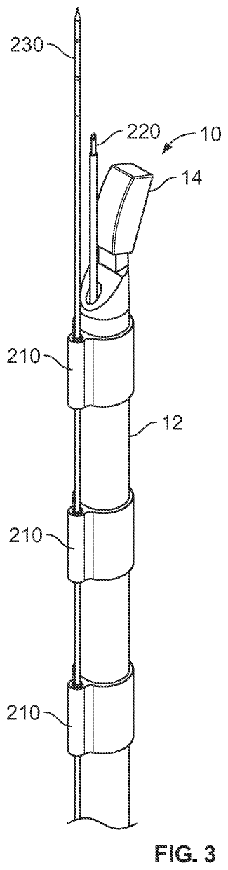

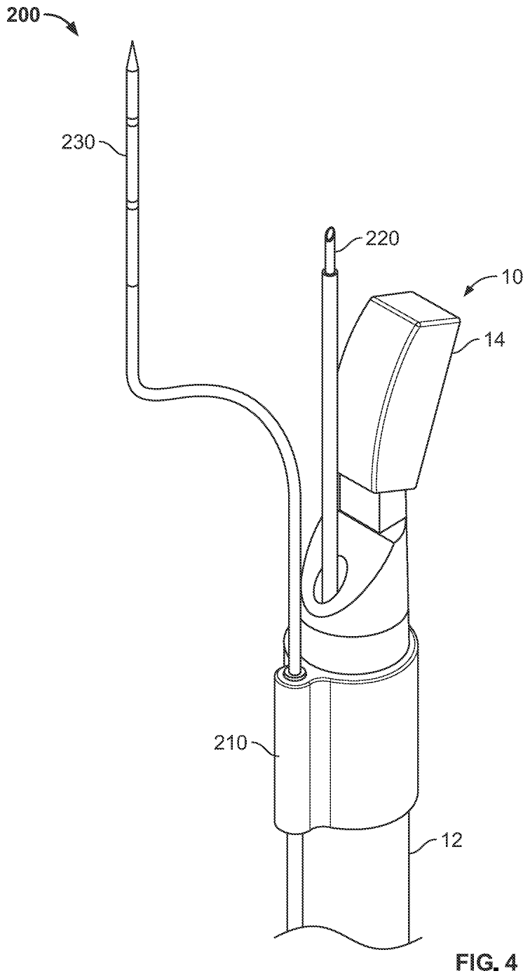

FIGS. 3 and 4 illustrate another example endoscopic tumor treatment system 200. Broadly speaking, the endoscopic tumor treatment system 200 includes an ultrasound probe 10, one or more collar adapters 210, an insulated needle 220, and an electrode lead 230.

The endoscopic tumor treatment system 200 can be navigated within a patient to a target location. For example, in some cases the endoscopic tumor treatment system 200 can be navigated within a trachea or bronchus of a patient to deliver therapy to tumors in mediastinal structures or through the gastrointestinal tract to deliver therapy in abdominal spaces. This can be performed while using visualization provided by the ultrasound probe 10.

The therapy delivered to tumors using the endoscopic tumor treatment system 200 can include at least two types of treatments. First, using the needle 220, processed tumor cells or immune response enhancing substances can be injected into the tumor. Second, using the electrode lead 230 as an anode and the needle 220 as a cathode, pulsed electrical field electroporation can be delivered to the tumor. In some cases, the pulsed electrical field electroporation can be non-thermal electroporation.

The ultrasound probe 10 comprises a shaft 12 and an ultrasonic transducer 14 attached to a distal end of the shaft 12. The shaft 12 defines at least one working channel. The shaft 12 and/or ultrasonic transducer 14 can be deflectable to facilitate steering and/or directing of the ultrasound probe 10.

In the depicted embodiment, the collar adapter 210 is slidably attached to the outer diameter of the shaft 12. In some embodiments, two or more collar adapters 210 can be included. The collar adapter 210 defines a lumen.

The electrode lead 230 is slidably disposed in the lumen defined by the collar adapter 210. The electrode lead 230 is a flexible, configurable lead that includes one or more contact electrodes that are spaced apart from each other. In some embodiments, three or more electrodes are included on the electrode lead 230. In some embodiments, the electrode lead 230 is a stylet-driven flexible lead that is configurable (e.g., steerable) in response to manipulation of an internal stylet. The contact electrodes on the electrode lead 230 can be activated simultaneously, in sequence, or in tandem to generate the desired electrical field.

The needle 220 is slidably disposed within the working channel of the shaft 12. The needle 220 includes a tip configured for puncturing tissue. The needle 220 defines a lumen that is used for obtaining core tissue or aspirates from the tumor, and/or for injecting processed tumor cells or other immune enhances into the tumor. The needle 220 is configured to be a cathode that functions in cooperation with the electrode lead 230 to deliver electroporation energy to a target tissue. In some embodiments, such as the depicted embodiment, an outer insulative covering is included on the needle 220 except for on a distal end portion of the needle 220. In other embodiments, the central need 220 can have multiple electrodes on its shaft to activation simultaneously or in sequence with the electrodes on the electrode lead 230 to create a desired immune enhancing electrical field.

FIG. 4, in particular, shows the endoscopic tumor treatment system 200 in a deployed state. In the deployed state, the electrode lead 230 and the needle 220 are both in position to contact tissue so that pulsed electrical field electroporation can be delivered to a target tissue (e.g., tumor, lesion, etc.) for therapeutic purposes. In addition, the needle 220 can be operated to obtain core tissue or aspirates from the tumor and/or for injecting processed tumor cells or other immune enhancing substances into the tumor. These operations can be performed under ultrasonic visualization using the ultrasound probe 10.

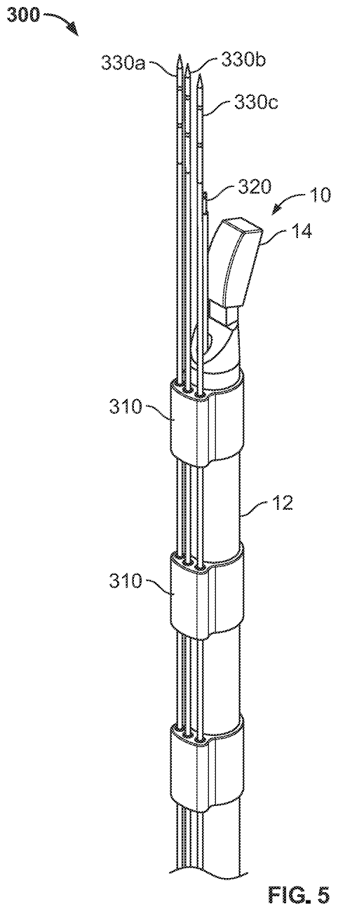

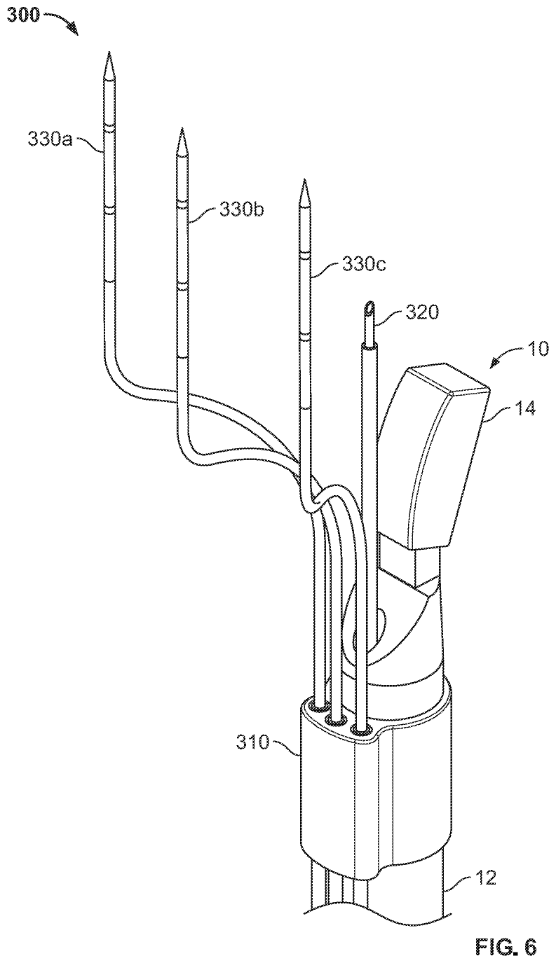

FIGS. 5 and 6 illustrate another example endoscopic tumor treatment system 300. Broadly speaking, the endoscopic tumor treatment system 300 includes an ultrasound probe 10, one or more collar adapters 310, an insulated needle 320, a first electrode lead 330a, a second electrode lead 330b, and a third electrode lead 330c.

The endoscopic tumor treatment system 300 can be navigated within a patient to a target location. For example, in some cases the endoscopic tumor treatment system 300 can be navigated within a trachea or bronchus of a patient to deliver therapy to tumors in mediastinal spaces or through the gastrointestinal tract to deliver treatments in abdominal spaces. This can be performed while using visualization provided by the ultrasound probe 10.

The therapy delivered to tumors using the endoscopic tumor treatment system 300 can include at least two types of treatments. First, using the needle 320, processed tumor cells or immune enhancers can be injected into the tumor. Second, using the electrode leads 330a-c as anodes and the needle 320 as a cathode, pulsed electrical field electroporation can be delivered to the tumor. In some cases, the pulsed electrical field electroporation can be non-thermal electroporation.

The ultrasound probe 10 comprises a shaft 12 and an ultrasonic transducer 14 attached to a distal end of the shaft 12. The shaft 12 defines at least one working channel. The shaft 12 and/or ultrasonic transducer 14 can be deflectable to facilitate steering and/or directing of the ultrasound probe 10.

In the depicted embodiment, the collar adapter 310 is slidably attached to the outer diameter of the shaft 12. In some embodiments, two or more collar adapters 310 can be included. The collar adapter 310 defines one or more lumens.

The electrode leads 330a-c are slidably disposed in the lumens defined by the collar adapter(s) 310. The electrode leads 330a-c are flexible, and or configurable, and or rotatable, and or steerable leads that include one or more contact electrodes that are spaced apart from each other. In some embodiments, three or more electrodes are included on each of the electrode leads 330a-c. In some embodiments, the electrode leads 330a-c are stylet-driven flexible leads that are configurable (e.g., steerable) in response to manipulation of internal stylets. The contact electrodes on 330a-c can be activated simultaneously, in sequence, or in tandem to generate the desired electrical field.

The needle 320 is slidably disposed within the working channel of the shaft 12. The needle 320 includes a tip configured for puncturing tissue. The needle 320 defines a lumen that is used for obtaining core tissue or aspirates from the tumor, and/or for injecting processed tumor cells or immunes enhancers into the tumor. The needle 320 is configured to be a cathode that functions in cooperation with the electrode leads 330a-c to deliver pulsed electrical field or electroporation energy to a target tissue. In some embodiments, such as the depicted embodiment, an outer insulative covering is included on the needle 320 except for on a distal end portion of the needle 320. In some embodiment the central needle 320 can have multiple electrodes on it shaft to enhance the multi-dimensional shape of the electrical field. In some embodiments, the central needle can also deliver adjunct ablation modalities to enhance immune activation such as cryoablation or radiofrequency ablation.

FIG. 6, in particular, shows the endoscopic tumor treatment system 300 in a deployed state. In the deployed state, the electrode leads 330a-c and the needle 320 are both in position to contact tissue so that pulsed electrical field electroporation can be delivered to a target tissue (e.g., tumor, lesion, etc.) for therapeutic purposes. In addition, the needle 320 can be operated to obtain core tissue from the tumor and/or for injecting processed tumor cells or immune enhancer into the tumor. These operations can be performed under ultrasonic visualization using the ultrasound probe 10.

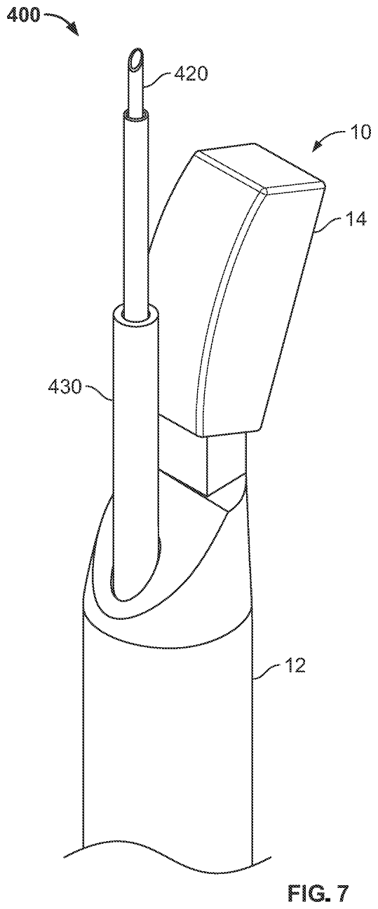

FIG. 7 illustrates another example endoscopic tumor treatment system 400. Broadly speaking, the endoscopic tumor treatment system 400 includes an ultrasound probe 10, an insulated needle 420, and an electrode 430.

The endoscopic tumor treatment system 400 can be navigated within a patient to a target location. For example, in some cases the endoscopic tumor treatment system 400 can be navigated within areas of the body of a patient such as, but not limited to, an esophagus, stomach, colon, small intestines, a trachea, or bronchus of the patient or in the gastrointestinal tract to deliver therapy to tumors in mediastinal and abdominal spaces. This can be performed while using visualization provided by the ultrasound probe 10.

The therapy delivered to tumors using the endoscopic tumor treatment system 400 can include at least two types of treatments. First, using the needle 420, processed tumor cells and or other immune enhancing substances such as monoclonal or multifunctional antibodies can be injected into the tumor. Second, using the electrode 430 as an anode and the needle 420 as a cathode, pulsed electrical field electroporation can be delivered to the tumor. In some cases, the pulsed electrical field electroporation can be non-thermal electroporation. In some embodiments, the central needle 420 can also deliver other adjunction ablation modalities such as cryoablation or radiofrequency ablation. In some embodiments, the ultrasound probe 10 be utilized for visualization, and can also deliver ultrasound, mechanical or focused waves to enhance further the generated immune response.

The ultrasound probe 10 comprises a shaft 12 and an ultrasonic transducer 14 attached to a distal end of the shaft 12. The shaft 12 defines at least one working channel. The shaft 12 and/or ultrasonic transducer 14 can be deflectable to facilitate steering and/or directing of the ultrasound probe 10.

The electrode 430 is slidably disposed within the working channel of the shaft 12. The electrode 430 defines a lumen, and is configured to be an anode.

The needle 420 is slidably disposed within the lumen of the electrode 430. The needle 420 includes a tip configured for puncturing tissue. The needle 420 defines a lumen that is used for obtaining core tissue from the tumor, and/or for injecting processed tumor cells into the tumor. The needle 420 is configured to be a cathode that functions in cooperation with the electrode 430 to deliver pulsed electrical field or electroporation energy to a target tissue. In some embodiments, such as the depicted embodiment, an outer insulative covering is included on the needle 420 except for on a distal end portion of the needle 420.

In the deployed state as depicted, the electrode 430 and the needle 420 are both in position to contact tissue so that pulsed electrical field electroporation can be delivered to a target tissue (e.g., tumor, lesion, etc.) for therapeutic purposes. For example, in the deployed state the electrode 430 and the needle 420 can be positioned in direct contact with a protruding tumor (e.g., airway compressive chest masses, etc.). In such a case, the endoscopic tumor treatment system 400 can be used to de-bulk or shrink the tumor. In addition, the needle 420 can be operated to obtain core tissue or aspirates from the tumor and/or for injecting processed tumor cells with other immune enhancing substance into the tumor. These operations can be performed under ultrasonic visualization using the ultrasound probe 10.

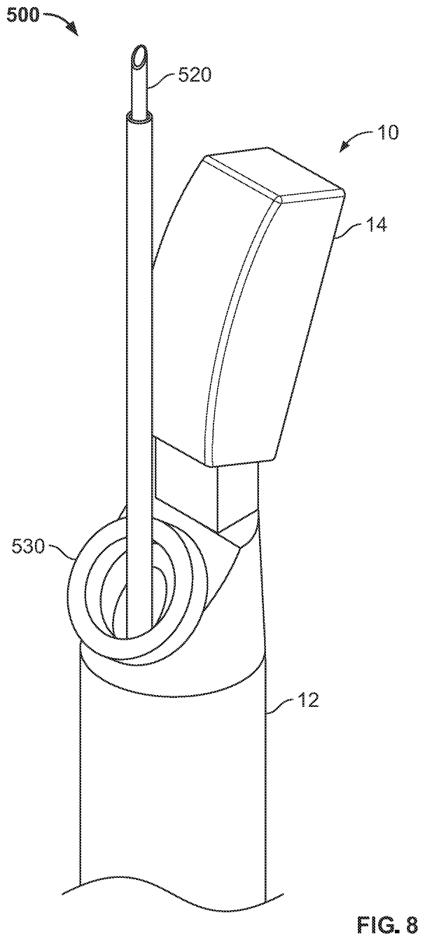

FIG. 8 illustrates another example endoscopic tumor treatment system 500. Broadly speaking, the endoscopic tumor treatment system 500 includes an ultrasound probe 10, an insulated needle 520, and an electrode 530.

The endoscopic tumor treatment system 500 can be navigated within a patient to a target location. For example, in some cases the endoscopic tumor treatment system 500 can be navigated within a trachea or bronchus of a patient to deliver therapy to tumors in mediastinal and abdominal spaces. This can be performed while using visualization provided by the ultrasound probe 10.

The therapy delivered to tumors using the endoscopic tumor treatment system 500 can include at least two types of treatments. First, using the needle 520, processed tumor cells or other immune enhancers can be injected into the tumor. Second, using the electrode 530 as an anode and the needle 520 as a cathode, pulsed electrical field electroporation can be delivered to the tumor. In some cases, the pulsed electrical field electroporation can be non-thermal electroporation. In some embodiments, the central needle 520 can have multiple electrodes on its shaft and/or deliver adjunct ablation modalities such as cryoablation or other types of ablation as described herein.

The ultrasound probe 10 comprises a shaft 12 and an ultrasonic transducer 14 attached to a distal end of the shaft 12. The shaft 12 defines at least one working channel. The shaft 12 and/or ultrasonic transducer 14 can be deflectable to facilitate steering and/or directing of the ultrasound probe 10.

The electrode 530 is attached to a distal end of the shaft 12. The electrode 530 defines an opening, and is configured to be a single anode or multiple anodes spread on it circumference.

The needle 520 is slidably disposed within the working channel of the shaft 12 and extends through the opening defined by the electrode 530. The needle 520 includes a tip configured for puncturing tissue. The needle 520 defines a lumen that is used for obtaining core tissue or aspirates from the tumor, and/or for injecting processed tumor cells or immune enhancers into the tumor. The needle 520 is configured to be a cathode that functions in cooperation with the electrode 530 to deliver pulsed electrical field or electroporation energy to a target tissue. In some embodiments, such as the depicted embodiment, an outer insulative covering is included on the needle 520 except for on a distal end portion of the needle 520. In some embodiments, that central needle 520 can have multiple electrodes on its shaft to deliver with 530 electrodes that desired electrical field for enhance immune response.

In the deployed state as depicted, the electrode 530 and the needle 520 are both in position to contact tissue so that pulsed electrical field electroporation can be delivered to a target tissue (e.g., tumor, lesion, etc.) for therapeutic purposes. For example, in the deployed state the electrode 530 and the needle 520 can be positioned in direct contact with a protruding tumor (e.g., airway compressive chest masses, etc.). In such a case, the endoscopic tumor treatment system 500 can be used to de-bulk or shrink the tumor. In addition, the needle 520 can be operated to obtain core tissue or aspirates from the tumor and/or for injecting processed tumor cells into the tumor. These operations can be performed under ultrasonic visualization using the ultrasound probe 10.

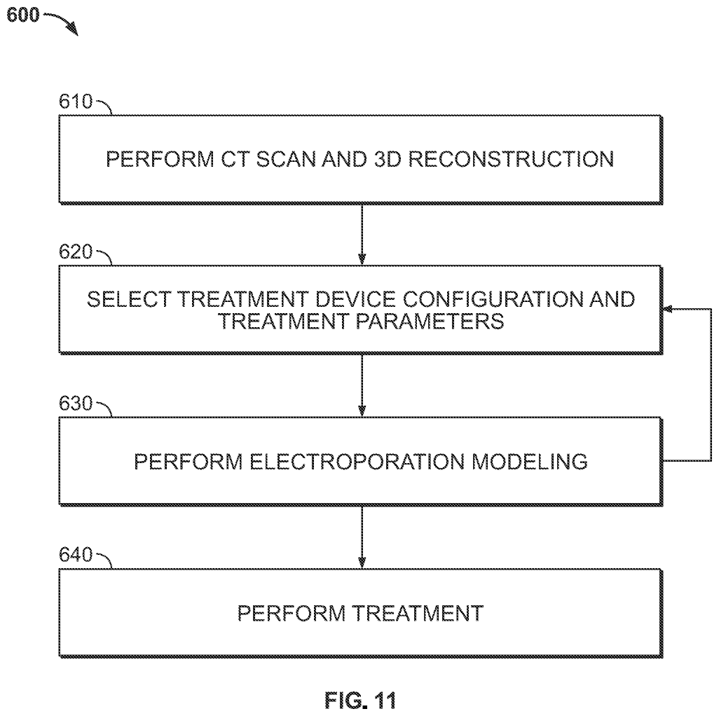

FIG. 11 illustrates a pre-procedure planning method 600 that can be used in conjunction with any of the endoscopic tumor treatment system described herein. The method 600 can be performed to help select the particular design and operational parameters of an endoscopic tumor treatment system that is best suited for a particular patient's condition. Accordingly, the efficacy of the endoscopic tumor treatment can be increased using the method 600.

In step 610, a CT (computed tomography) scan or other cross section modality or imaging registration system and 3D reconstruction of the target area of the patient to be treated can be performed. For example, in some cases the CT scan and 3D reconstruction can capture and illustrate the position, size, and orientation of one or more tumors to be targeted in relation to the surrounding anatomy of the patient.

In step 620, initial selections of the endoscopic tumor treatment system configuration and treatment parameters can be made based on the CT scan or other cross section modality or image registration system and 3D reconstruction. This selection can include, for example, numbers of electrodes, positions to penetrate the tumor(s), spatial orientation and angel of deflection, and the energy parameters (e.g., voltage, pulse parameters, frequency, etc.) to delivery the desired electrical field for maximal therapeutic effect.

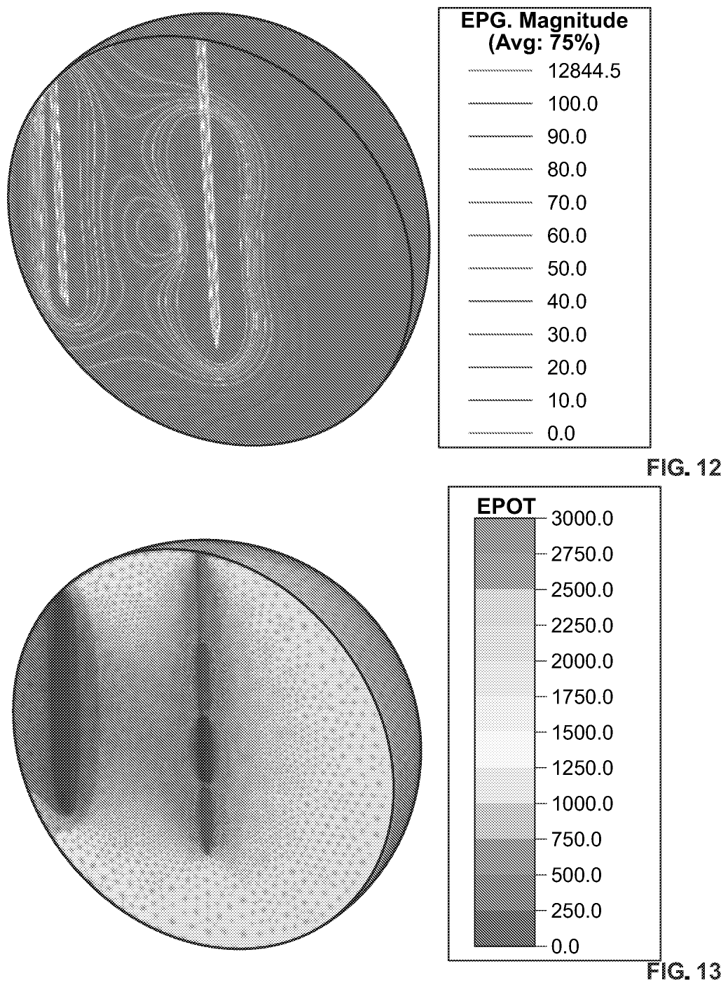

In step 630, electroporation modeling is performed based on the endoscopic tumor treatment system configuration and treatment parameters selected in step 620. FIGS. 12 and 13 illustrate examples of electroporation modeling. The electroporation modeling illustrates the field strength of the electroporation energy at various locations within the tumor(s), for example. The electroporation modeling also illustrates the potential affects to surrounding anatomical structures such as muscles, blood vessels, bones, and the like.

If the electroporation modeling in step 630 is satisfactory, the treatment plan is complete and the treatment can be performed in step 640. However, if the electroporation modeling in step 630 is unsatisfactory, the method 600 can revert to step 620 for modifications to the selections of the tumor treatment system configuration and treatment parameters. Thereafter, the method 600 can again proceed to step 630 for another iteration of the electroporation modeling. Again, if the results of the reiteration of the electroporation modeling are satisfactory, the treatment plan is complete and the treatment can be performed in step 640. Otherwise, further improvements to the tumor treatment system configuration and treatment parameters can again be selected in step 620. Eventually, the treatment is performed in step 640.

### Additional Features and Embodiments

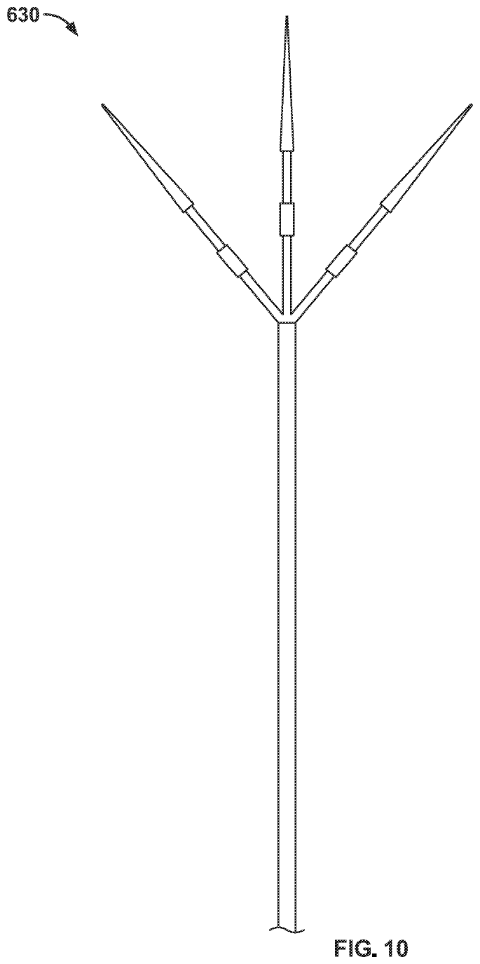

FIG. 10 illustrates an alternative style of electrode 630. The electrode 630 can be used with any of the endoscopic tumor treatment systems described herein.

The electrode 630 includes a distal end portion that divides into multiple branches on which one or more electrodes are positioned. In the depicted embodiment, the electrode 630 includes three electrode branches. In some embodiments, two electrode branches, four electrode branches, or more than four electrode branches are included. Such electrodes can be used to deliver electroporation energy and/or for performing impedance measurements.

While the main electrode shaft and the electrode branches of the illustrated electrode 630 are linear, in some embodiments any or all of the main electrode shaft and/or the electrode branches can have natural curves, angles, or any desired shape. In some embodiments, each of the electrode branches can have a stylet therein by which the shape of the electrode branches can be controlled.

In some embodiments, the endoscopic tumor treatment systems described herein can include locking mechanisms that can be selectively activated to secure the proximal/distal locations of the electrodes and/or needles relative to the sheath. Moreover, in some embodiments a locking mechanism can be included that can be activated to secure the proximal/distal location of the sheath relative to the shaft 12. In some embodiments, such a locking mechanism can be a physical mechanism (e.g., a friction-applying member, a pawl latch, a projection and indent mechanism, a spring mechanism, etc.). In some embodiments, such a locking mechanism can be a magnetic locking mechanism (e.g., an electromagnetic device that can be selectively activated). The locking mechanisms can be activated by a clinician operator at the handle of the endoscopic tumor treatment systems.

In some embodiments, magnets (e.g., permanent magnets, electromagnets, and combinations thereof) can be used to steer the advancement of the electrodes and/or needles. For example, in some embodiments a magnet can be used on the skin surface of the patient to control the positioning of the electrodes and/or needles within the body of the patient.

It should be understood that any combination of the features of any of the embodiments described herein can be combined with any of the features of the other embodiments described herein. Accordingly, hybrid designs of the endoscopic tumor treatment systems described herein can be created and are within the scope of this disclosure.

While this specification contains many specific implementation details, these should not be construed as limitations on the scope of any invention or of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described herein as acting in certain combinations and even initially claimed as such, one or more features from a claimed combination can in some cases be excised from the combination, and the claimed combination may be directed to a subcombination or variation of a subcombination.

Similarly, while operations may be described in a particular order, this should not be understood as requiring that such operations be performed in the particular order shown or in sequential order, or that all illustrated operations be performed, to achieve desirable results.

Particular embodiments of the subject matter have been described. Other embodiments are within the scope of the following claims. For example, the actions recited in the claims can be performed in a different order and still achieve desirable results.

1.-17. (canceled)

18. An endoscopic or endobronchial tumor treatment system comprising:
an ultrasound probe comprising a shaft and an ultrasonic transducer attached to a distal end of the shaft, the shaft defining a working channel;
a sheath defining a first lumen and a second lumen, the sheath being slidably disposable within the working channel and extendable from a distal end of the working channel;
a needle slidably disposed within the first lumen, the needle defining a lumen and configured to be a cathode; and
an electrode lead slidably disposed within the second lumen and configured to be an anode.

19. The system of claim 18, wherein the electrode lead is a stylet-driven flexible lead that is configurable by an internal stylet.

20. The system of claim 18, wherein the electrode lead comprises three or more contact electrodes.

21. The system of claim 18, wherein the needle is configured to obtain a tumor tissue sample and to inject processed tissue or other immune enhancing substances into a tumor.

22. The system of claim 18, wherein the sheath is distally extendable from a distal end of the working channel.

23. The system of claim 18 wherein the needle is configured to deliver a pulsed electrical field or electroporation energy to a target tissue.

24. The system of claim 18, wherein the ultrasound probe is configured to provide ultrasonic visualization for endoscopically or endobronchially treating tumors.

25. An endoscopic or endobronchial tumor treatment system comprising:
an ultrasound probe comprising a shaft and an ultrasonic transducer attached to a distal end of the shaft, the shaft defining a working channel;
a needle slidably disposed within the working channel, the needle defining a lumen and configured to be a cathode;
a collar adapter attached to the shaft of the endoscope and defining a lumen; and
an electrode lead comprising one or more contact electrodes and being slidably disposed within the lumen of the collar adapter and configured to be an anode.

26. The system of claim 25, wherein further comprising an outer insulative covering on the needle except for on a distal end portion of the needle.

27. The system of claim 25, wherein the electrode lead is a stylet-driven flexible lead that is configurable by an internal stylet.

28. The system of claim 25, wherein the electrode lead comprises three or more contact electrodes.

29. The system of claim 25, wherein the needle is configured to obtain a tumor tissue sample and to inject processed tissue or other immune enhancing substances into a tumor.

30. The system of claim 25 wherein the collar adapter is slidably attached to the outer diameter of the shaft of the endoscope.

31. The system of claim 25, wherein the electrode lead and the needle are positioned to contact tissue when configured in a deployed state.

32. The system of claim 25, wherein the needle is configured to deliver a pulsed electrical field or electroporation energy to a target tissue.

33. An endoscopic or endobronchial tumor treatment system comprising:
an ultrasound probe comprising a shaft and an ultrasonic transducer attached to a distal end of the shaft, the shaft defining a working channel;
a needle slidably disposed within the working channel, the needle defining a lumen and configured to be a cathode;
a collar adapter attached to the shaft of the endoscope and defining a first lumen, a second lumen, and a third lumen;
a first electrode lead slidably disposed within the first lumen of the collar adapter and configured to be an anode;
a second electrode lead slidably disposed within the second lumen of the collar adapter and configured to be an anode; and
a third electrode lead slidably disposed within the third lumen of the collar adapter and configured to be an anode.

34. The system of claim 33, wherein further comprising an outer insulative covering on the needle except for on a distal end portion of the needle.

35. The system of claim 33, wherein the first, second, and third electrode leads are each stylet-driven flexible leads that are configurable by an internal stylet.

36. The system of claim 33, wherein the first, second, and third electrode leads each comprise three or more contact electrodes.

37. The system of claim 33, wherein the needle is configured to obtain a tumor tissue sample and to inject processed tissue or other immune enhancing substances into a tumor.
